(19) United States
(12) Patent Application Publication
(10) Pub. No.: US 2026/0308632 A1
(43) Pub. Date: Oct. 8, 2026

(54) CLICK CHEMISTRY ASSISTED CELL SURFACE ENGINEERING FOR RESTORING LENTIVIRUS ENTRY

(71) Applicants: THE CHILDREN'S HOSPITAL OF PHILADELPHIA, Philadelphia, PA (US); THE TRUSTEES OF THE UNIVERSITY OF PENNSYLVANIA, Philadelphia, PA (US)

(72) Inventors: Leyuan MA, Philadelphia, PA (US); Letitia CHAN, Philadelphia, PA (US)

(73) Assignees: THE CHILDREN'S HOSPITAL OF PHILADELPHIA, Philadelphia, PA (US); THE TRUSTEES OF THE UNIVERSITY OF PENNSYLVANIA, Philadelphia, PA (US)

(21) Appl. No.: 19/479,881

(22) PCT Filed: Jun. 28, 2024
(86) PCT No.: PCT/US2024/036012

§ 371 Date: Oct. 29, 2025

Related U.S. Application Data
(60) Provisional application No. 63/511,490, filed on Jun. 30, 2023.

(51) Int. Cl. C12N 15/867; C07K 14/705; C07K 16/10; C12N 5/0783; C12N 5/0789; C12N 5/09

(52) U.S. Cl. CPC C12N 15/867; C07K 14/705; C07K 16/10; C12N 5/0636; C12N 5/0646; C12N 5/0647; C12N 5/0693; C12N 2510/00; C12N 2740/15043

(57) ABSTRACT

The present disclosure is directed to methods of labeling the surface of cells with a ligand for improved transduction. The cells may be labeled with a recombinant LDLR or anti-VSV-G antibody, such as by click chemistry. Further provided herein are methods of gene therapy, screening, and disease models using the present surface labeled cells.

### PRIORITY CLAIM

This application claims benefit of priority to U.S. Provisional Application Ser. No. 63/511,490, filed Jun. 30, 2023, the entire contents of which are hereby incorporated by reference.

### STATEMENT REGARDING FEDERALLY FUNDED RESEARCH

This invention was made with government support under grant number AI164319 awarded by the National Institutes of Health. The government has certain rights in the invention.

### BACKGROUND

### 1. Field of the Disclosure

The present disclosure relates generally to the fields of molecular biology. More particularly, the disclosure relates to surface labeled cells for improved transduction, such as for gene therapy.

### 2. Background

In current laboratory practice, transduction efficiency of certain cell types using VSV-G pseudotyped lentiviruses is quite low. For example, in murine T cells the transduction efficiency ranges from 5-10%. In canine hematopoietic cells, there is also low transduction efficiency, typically less than 1%. Given that many CRISPR libraries and genetic engineering tools are all lentivirus-based, it is extremely impactful to expand the application of these tools to a wider variety of cell types and animal models for immunology research as well as pre-clinical evaluation of cell-based immunotherapies. Thus, there is an unmet need for improved methods of lentivirus transduction.

### SUMMARY

Thus, in accordance with the present disclosure, there is a method of chemically labeling a cell comprising attaching to the surface of said cell a recombinant low density lipoprotein receptor (LDLR) protein or anti-vesicular stomatitis virus envelope glycoprotein (VSV-G) antibody or fragment thereof by performing click chemistry.

In some aspects, the cell has low, undetectable or essentially no expression of LDLR, such as by measuring with flow cytometry with an anti-LDLR antibody. In some aspects, the anti-VSV-G antibody is I1 antibody. In certain aspects, performing click chemistry comprises:
(a) conjugating Sulfo-NHS DBCO to recombinant LDLR or anti-VSV-G antibody to obtain DBCO-labeled protein;
(b) conjugating azide to cell surface proteins of said cell using 6-azidohexanoic acid-NHS ester to obtain an azide-labeled cell; and
(c) mixing the DBCO-protein with the azide-labeled cell to obtain a LDLR or anti-VSV-G antibody labeled cell.

In some aspects, the mixing is performed at 0.1 mg/mL. In certain aspects, the cell with low or essentially no expression of hLDLR is a human naïve T cell, a resting human T cell, a human B cells, a human epithelial cell, a murine immune cell, a canine immune cell, a monkey immune cell, or a cancer cell line.

In certain aspects, the murine immune cell or canine immune cell is a T cell, NK cell, or hematopoietic cell. In some aspects, the murine immune cell, monkey immune cell, or canine immune cell is a T cell. In certain aspects, the cancer cell line is SUP-B15 or Raji. In some aspects, the method further comprises transducing the cell with lentivirus. In certain aspects, the lentivirus comprises VSV-G as the envelope protein. In some aspects, transduction efficiency is at least 80%.

A further embodiment provides a chemically labeled cell having a recombinant LDLR or anti-VSV-G antibody or fragment thereof attached to the surface. In some aspects, the cell is produced by the present embodiments and aspects thereof (e.g., a method of chemically labeling a cell comprising attaching to the surface of said cell a recombinant low density lipoprotein receptor (LDLR) protein or anti-vesicular stomatitis virus envelope glycoprotein (VSV-G) antibody or fragment thereof by performing click chemistry). In certain aspects, the cell has been transduced by a VSV-G lentivirus.

Further embodiments provide use of the present chemically labeled cell having a recombinant LDLR or anti-VSV-G antibody or fragment thereof attached to the surface for gene therapy, lentivirus-based in vitro gene delivery, human naïve T cell engineering, and genetic screens using murine T cells for T cell immunology and cancer immunology research.

In additional embodiments, the present lentivirus can be used for pre-clinical studies using the customized lentiviruses or a lentivirus library for genomic screens (e.g., RNAi, CRISPR, ORF library). For example, the desired non-transducible cells can be surface-labeled with fluorescein followed by the addition of VSVG/aFITC dual-envelope pseudotyped lentivirus using routine transduction method (e.g., spin transduction). Further embodiments provide methods for using the present lentivirus for pre-clinical studies using a gene-modified mouse, canine, or monkey T cells. Another embodiment provides use of naïve, resting or activated human T cells pre-labeled with fluorescein followed by addition of VSVG/aFITC dual envelope pseudotyped lentivirus using routine transduction method (e.g., spin transduction) for gene therapy.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1: Schematics lentiviral transduction of the cells with or without hLDLR expression and surface engineering approach to promote standard lentiviral transduction.

FIG. 2: Lentiviral transduction of WT and LDLR-deficient K562 cells.

FIGS. 3A-3C: Lentiviral transduction of WT and LDLR-deficient K562 cells with or without click-chemistry assisted surface attachment of recombinant LDLR or I1 antibody. Shown on the left are the representative scatter plot, shown on the right are the summary plots. **p<0.0001, ***p<0.001, **p<0.01.

### DESCRIPTION OF ILLUSTRATIVE EMBODIMENTS

Lentiviral transduction is a commonly used technology in modern laboratories and clinical therapies due to their safe and stable integration of genes into human cell genomes. The human Low Density Lipoprotein Receptor (hLDLR) has recently been identified as the natural target of VSV-G and serves as the entry receptor for VSV-G pseudotyped lentiviral vectors. However, many cell types have poor transducibility by the most widely used VSV-G pseudotyped lentiviral vectors, mostly due to the lack of expression of the proper lentiviral entry receptor, hLDLR. These include murine immune cell types, such as T cells, canine immune cells, and also human naïve T cells, certain epithelial cells, and more. The present disclosure provides methods to overcome this limitation in these cell types with a broad method to increase the efficiency of transduction. It was shown that in cells without hLDLR expression, chemical labelling resulted in increased lentiviral transduction ex vivo, using pseudotyped lentivirus.

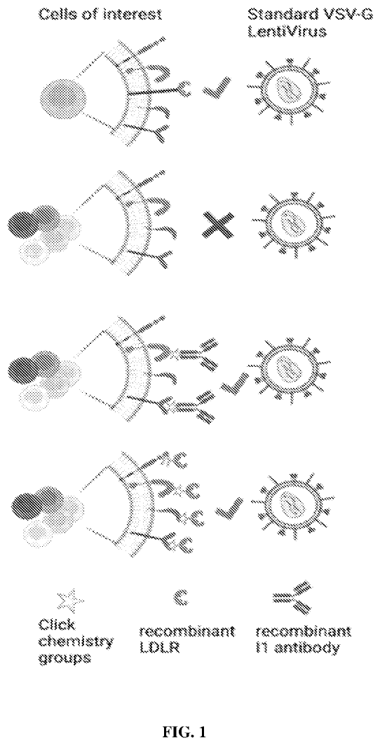

In the present studies, a strategy was developed to chemically label cell surfaces with a ligand (i.e., recombinant LDLR or I1 antibody (anti-VSV-G)) that can be recognized by VSV-G, priming them for docking and lentivirus entry. These cells can then be efficiently transduced by the standard lentiviruses that contain VSV-G as the envelope protein. This strategy was found to dramatically improve lentiviral transduction efficiency (FIG. 1).

Further provided herein are use of the present transduction strategy for gene therapy and lentivirus-based in vitro gene delivery, human naïve T cell engineering, and genetic screens using murine T cells for T cell immunology and cancer immunology research. The present methods can be used to significantly accelerate the development of various disease models using lentivirus-based disease induction.

In additional embodiment, the present strategy can be used in both the academic labs and industry to perform genetic screens in immune cells, especially T cells, for identifying therapeutic target to improve immunotherapy, to assist in the development and evaluation of gene and cell therapy across cell types and species, and to assist in lentiviral engineering and targeted in vivo gene delivery if a bispecific engager is used, e.g., I1 scFV-anti-CD3 for T cell directed gene delivery.

These and other aspects of the disclosure are described in detail below.

### I. Cell Surface Labeling

Existing methods of transducing murine T cells with VSV-G based lentivirus largely experience low transduction efficiency, ranging from 5-10%. In canine hematopoetic cells, there is also low transduction efficiency, typically less than 1%, and improvements in these cell types have only raised transduction to approximately 12%. In these cell types, and other non-transducible cell types, this is mostly due to the lack of an optimal entry receptor, hLDLR, for the lentivirus particles. Manipulation of primary T cells with specific culturing methods, which is the current method to improve transduction, to improve lentiviral transduction likely lead to undesired cell phenotypes.

In certain embodiments, the present methods provide a strategy of chemically labeling cells to restore lentivirus binding with recombinant LDLR or recombinant I1 antibody (anti-VSV-G) which offers a simple platform to achieve higher efficiency lentiviral transduction with high applicability and flexibility towards various cell types. Chemical labelling of target cell surface with recombinant proteins does not alter the cell genotype, and this approach is compatible with standard lentiviral methods commonly used across the world in both academic and industrial settings. This strategy overcomes the limitation of hLDLR entirely, providing the potential for lentiviral-based transduction in animal cells such as murine and canine cells, as well as nontransducible human cell types, such as naïve T cells. In some aspects, the cell is labeled with the recombinant LDLR or anti-VSV-G antibody, such as I1 antibody, by click chemistry. Alternative clones of anti-VSV-G monoclonal antibody can be used, including M55, 8G5F11 and IE9F9. Polyclonal antibodies can also be used.

The low-density lipoprotein receptor (LDL-R) is a mosaic protein of 839 amino acids (after removal of 21-amino acid signal peptide) that mediates the endocytosis of cholesterol-rich low-density lipoprotein (LDL). It is a cell-surface receptor that recognizes apolipoprotein B100 (ApoB100), which is embedded in the outer phospholipid layer of very low-density lipoprotein (VLDL), their remnants—i.e. intermediate-density lipoprotein (IDL), and LDL particles. The receptor also recognizes apolipoprotein E (ApoE) which is found in chylomicron remnants and IDL. In humans, the LDL receptor protein is encoded by the LDLR gene on chromosome 19. It belongs to the low-density lipoprotein receptor gene family. It is most significantly expressed in bronchial epithelial cells and adrenal gland and cortex tissue.

Disruption of LDL-R can lead to higher LDL-cholesterol as well as increasing the risk of related diseases. Individuals with disruptive mutations (defined as nonsense, splice site, or indel frameshift) in LDLR have an average LDL-cholesterol of 279 mg/dl, compared with 135 mg/dL for individuals with neither disruptive nor deleterious mutations. Disruptive mutations were 13 times more common in individuals with early-onset myocardial infarction or coronary artery disease than in individuals without either disease.

The human LDLR gene resides on chromosome 19 at the band 19p13.2 and is split into 18 exons. Exon 1 contains a signal sequence that localizes the receptor to the endoplasmic reticulum for transport to the cell surface. Beyond this, exons 2-6 code the ligand binding region; 7-14 code the epidermal growth factor (EGF) domain; 15 codes the oligosaccharide rich region; 16 (and some of 17) code the membrane spanning region; and 18 (with the rest of 17) code the cytosolic domain. This gene produces 6 isoforms through alternative splicing.

This protein belongs to the LDLR family and is made up of a number of functionally distinct domains, including 3 EGF-like domains, 7 LDL-R class A domains, and 6 LDL-R class B repeats. Representative sequences can be found at NP_000518 (protein) and NP_000527 (mRNA).

The N-terminal domain of the LDL receptor, which is responsible for ligand binding, is composed of seven sequence repeats (~50% identical). Each repeat, referred to as a class A repeat or LDL-A, contains roughly 40 amino acids, including 6 cysteine residues that form disulfide bonds within the repeat. Additionally, each repeat has highly conserved acidic residues which it uses to coordinate a single calcium ion in an octahedral lattice. Both the disulfide bonds and calcium coordination are necessary for the structural integrity of the domain during the receptor's repeated trips to the highly acidic interior of the endosome. The exact mechanism of interaction between the class A repeats and ligand (LDL) is unknown, but it is thought that the repeats act as “grabbers” to hold the LDL. Binding of ApoB requires repeats 2-7 while binding ApoE requires only repeat 5 (thought to be the ancestral repeat).

Next to the ligand binding domain is an EGF precursor homology domain (EGFP domain). This shows approximately 30% homology with the EGF precursor gene. There are three “growth factor” repeats; A, B and C. A and B are closely linked while C is separated by the YWTD repeat region, which adopts a beta-propeller conformation (LDL-R class B domain). It is thought that this region is responsible for the pH-dependent conformational shift that causes bound LDL to be released in the endosome.

A third domain of the protein is rich in O-linked oligosaccharides but appears to show little function. Knockout experiments have confirmed that no significant loss of activity occurs without this domain. It has been speculated that the domain may have ancestrally acted as a spacer to push the receptor beyond the extracellular matrix. The single transmembrane domain of 22 (mostly) non-polar residues crosses the plasma membrane in a single alpha helix. The cytosolic C-terminal domain contains ~50 amino acids, including a signal sequence important for localizing the receptors to clathrin-coated pits and for triggering receptor-mediated endocytosis after binding. Portions of the cytosolic sequence have been found in other lipoprotein receptors, as well as in more distant receptor relatives.

A click-chemistry based method can be used for a two-step labeling procedure for labeling cells with LDLR or anti-VSV-G antibody. In an exemplary method, dibenzocyclooctyne (DBCO) may be randomly conjugated to the recombinant LDLR or anti-VSV-G antibody using Sulfo-NHS DBCO. Azide may be conjugated to the cell surface proteins using 6-azidohexanoic acid-NHS ester (FIG. 1). The proteins can then be clicked to the cells by mixing the DBCO-labeled protein with Azide-labeled cells, such as by mixing them at 0.1 mg/ml and incubating at 37° C. for 30 min.

“Click chemistry” as utilized in this specification is defined as a chemical reaction involving molecular building blocks that selectively and covalently bond or “click” together. A “cycloaddition” reaction as utilized in this specification is defined as a type of click chemistry reaction. One embodiment of the present disclosure utilizes a 1+3-dipolar cycloaddition reaction of azide and alkyne functional groups, otherwise referred to as a [3+2]cycloaddition reaction. Other embodiments may involve other reactions including, for example, the Diels-Alder [4+2] cylcoaddition reaction between a diene and a dienophile. Examples of chemical linking modules include azide/alkyne, azide/DBCO, tetrazine/TCO, aldehyde/oxyamine, etc. Click chemistry pairs are a favorable choice since they are bio-orthoganol and highly efficient.

Carolyn Bertozzi and coworkers discovered copper-free click chemistry (formally known as strain-promoted azide-alkyne cycloaddition, or SPAAC) in 2004. Bertozzi was looking for bioconjugation techniques that were both bioorthogonal and biocompatible. The copper-catalyzed azide-alkyne cycloaddition (CuAAC) reaction independently discovered in 2002 by Meldal's group and Sharpless and coworkers uses copper(I) salts that are often toxic to live cells. The cytotoxicity of these copper salts spurred the development of less toxic and non-toxic alternatives that are biocompatible.

Dibenzocyclooctyne(DBCO) reagent is a class of click chemistry labeling reagents. DBCO group can exclusively react with azide-tagged molecules or biomolecules to form a stable triazole. Click chemistry is also known as strain promoted alkyne-azide cycloaddition (SPAAC), DBCO reagent has become widely used in bioconjugation, labeling and chemical biology. DBCO click chemistry can be run in aqueous buffer or in organic solvents depending on the property of the substrate molecules. Reagents with PEG arm will increase the compound's hydrophilicity. This DBCO-Azide method requires to activate the first molecule with DBCO reagent, and the second molecule with azide, then to mixing the two activated molecules to form a conjugate.

### II. Lentivirus Transduction

The chemically labeled cells of the present disclosure can further be transduced with a lentivirus, such as a lentivirus comprising VSV-G as the envelope protein. Thus, further provided herein are nucleic acids encoding a lentivirus and vectors comprising said nucleic acids.

Nucleic acid molecules, which are also referred to herein as polynucleotides or nucleic acid sequences, include DNA, such as cDNA or genomic DNA, and RNA. It is understood that the term “RNA” as used herein comprises all forms of RNA including mRNA, tRNA and rRNA but also genomic RNA, such as in case of RNA of RNA viruses. In particular, embodiments reciting “RNA” are directed to mRNA. Further included are nucleic acid mimicking molecules known in the art such as synthetic or semi-synthetic derivatives of DNA or RNA and mixed polymers, both sense and antisense strands. They may contain additional non-natural or derivatized nucleotide bases, as will be readily appreciated by those skilled in the art. Such nucleic acid mimicking molecules or nucleic acid derivatives according to the present disclosure include peptide nucleic acid (PNA), phosphorothioate nucleic acid, phosphoramidate nucleic acid, 2′-O-methoxyethyl ribonucleic acid, morpholino nucleic acid, hexitol nucleic acid (HNA) and locked nucleic acid (LNA), an RNA derivative in which the ribose ring is constrained by a methylene linkage between the 2′-oxygen and the 4′-carbon (see, for example, Braasch and Corey, Chemistry & Biology 8, 1-7 (2001)). PNA is a synthetic DNA-mimic with an amide backbone in place of the sugar-phosphate backbone of DNA or RNA, as described by Nielsen et al., Science 254:1497(1991 ); and Egholm et al., Nature 365:666 (1993).

The term “(poly)peptide” as used herein relates to polypeptides as well as peptides. The term “polypeptide”, as used herein interchangeably with the term “protein”, describes linear molecular chains of amino acids, including single chain proteins or their fragments, containing more than 30 amino acids, whereas the term “peptide” as used herein describes a group of molecules consisting of up to 30 amino acids. (Poly)peptides may further form oligomers consisting of at least two identical or different molecules. The corresponding higher order structures of such multimers are, correspondingly, termed homo-or heterodimers, homo- or heterotrimers etc. Such multimers also fall under the definition of the term “(poly)peptide”. The terms “polypeptide” and “peptide” also refer to naturally modified polypeptides/peptides where the modification is achieved, e.g., by glycosylation, acetylation, phosphorylation and similar modifications which are well known in the art.

The present vector may be a plasmid, cosmid, virus, bacteriophage or another vector used, e.g., conventionally in genetic engineering. The nucleic acid molecules provided herein may be inserted into several commercially available vectors suitable for the expression of eukaryotic proteins. Non-limiting examples include prokaryotic plasmid vectors, such as the pUC-series, pBluescript (Stratagene), the pET-series of expression vectors (Novagen) or pCRTOPO (Invitrogen) and vectors compatible with an expression in mammalian cells like pREP (Invitrogen), pcDNA3 (Invitrogen), pCEP4 (Invitrogen), pMC1neo (Stratagene), pXT1 (Stratagene), pSG5 (Stratagene), EBO-pSV2neo, pBPV-1, pdBPVMMTneo, pRSVgpt, pRSVneo, pSV2-dhfr, pIZD35, pLXIN, pSIR (Clontech), pIRES-EGFP (Clontech), pEAK-10 (Edge Biosystems) pTriEx-Hygro (Novagen) and pCINeo (Promega).

The present nucleic acid molecules may also be inserted into vectors such that a translational fusion with another polynucleotide is generated. The other polynucleotide may encode a protein which may, e.g., increase the solubility and/or facilitate the purification of the protein. Non-limiting examples include pET32, pET41, pET43.

For vector modification techniques, see Sambrook and Russell “Molecular Cloning, A Laboratory Manual”, Cold Spring Harbor Laboratory, N.Y. (2001). Generally, vectors can contain one or more origin of replication (ori) and inheritance systems for cloning or expression, one or more markers for selection in the host, e.g., antibiotic resistance, and one or more expression cassettes. Suitable origins of replication (ori) include, for example, the Col E1, the SV40 viral and the M 13 origins of replication.

The coding sequences inserted in the vector can, e.g., be synthesized by standard methods, or isolated from natural sources. Ligation of the coding sequences to transcriptional regulatory elements and/or to other amino acid encoding sequences can be carried out using established methods. Transcriptional regulatory elements (parts of an expression cassette) ensuring expression of the coding sequences are well known to those skilled in the art. These elements comprise regulatory sequences ensuring the initiation of the transcription (e. g., translation initiation codon, promoters, enhancers, and/or insulators), internal ribosomal entry sites (IRES) (Owens, Proc. Natl. Acad. Sci. USA 98 (2001), 1471-1476) and optionally poly-A signals ensuring termination of transcription and stabilization of the transcript. Additional regulatory elements may include transcriptional as well as translational enhancers, and/or naturally associated or heterologous promoter regions. In particular, the nucleic acid molecule of the present disclosure is operatively linked to such expression control sequences allowing its expression. The vector may further comprise nucleotide sequences encoding secretion signals as further regulatory elements. Such sequences are well known to those skilled in the art. Furthermore, depending on the expression system used, leader sequences capable of directing the expressed polypeptide to a cellular compartment may be added to the coding sequence of the polynucleotide of the present disclosure. Such leader sequences are well known in the art.

Possible examples for regulatory elements ensuring the initiation of transcription comprise the cytomegalovirus (CMV) promoter, SV40-promoter, RSV-promoter (Rous sarcome virus), the lacZ promoter, the gai10 promoter, human elongation factor 1α-promoter, CMV enhancer, CaM-kinase promoter, the Autographa californica multiple nuclear polyhedrosis virus (AcMNPV) polyhedral promoter or the SV40-enhancer. For the expression in prokaryotes, a multitude of promoters including, for example, the tac-lac-promoter, the lacUV5 or the trp promoter, has been described. Examples for further regulatory elements in prokaryotes and eukaryotic cells comprise transcription termination signals, such as SV40-poly-A site or the tk-poly-A site or the SV40, lacZ and AcMNPV polyhedral polyadenylation signals, downstream of the polynucleotide.

Furthermore, the present vectors may comprise a selectable marker. Examples of selectable markers include neomycin, ampicillin, hygromycin resistance and the like. Specifically designed vectors allow the shuttling of DNA between different hosts, such as bacteria-fungal cells or bacteria-animal cells.

An expression vector as used herein is capable of directing the replication, and the expression, of the nucleic acid molecule and encoded protein. Suitable expression vectors which comprise the described regulatory elements are known in the art such as pGreenPuro (System Biosciences, Mountain View, Calif., USA), pRc/CMV, pcDNA1, pcDNA3 (In-Vitrogene, as used, inter alia in the appended examples), pSPORT1 (GIBCO BRL) or pGEMHE (Promega), or prokaryotic expression vectors, such as lambda gt11, pJOE, the pBR1-MCS-series.

The nucleic acid molecules of the present disclosure as described herein above may be designed for direct introduction or for introduction via liposomes, phage vectors or viral vectors (e.g., adenoviral, retroviral) into the cell. Additionally, baculoviral systems or systems based on Vaccinia Virus or Semliki Forest Virus can be used as eukaryotic expression system for the nucleic acid molecules of the present disclosure.

The present disclosure further relates to a host cell comprising the nucleic acid molecule or the vector provided herein. Suitable prokaryotic hosts comprise, e.g., bacteria of the species Escherichia, Streptomyces, Salmonella or Bacillus. Suitable eukaryotic host cells are, e.g., yeasts such as Saccharomyces cerevisiae, Pichia pastoris, Schizosaccharomyces pombe or chicken cells, such as, e.g., DT40 cells. Insect cells suitable for expression are, e.g., Drosophila S2, Drosophila Kc, or Spodoptera Sf9 and Sf21 cells. Suitable zebrafish cell lines include, without being limiting, ZFL, SJD or ZF4.

Mammalian host cells that could be used include, human Hela, HEK293, HEK293T, H9 and Jurkat cells, mouse NIH3T3 and C127 cells, COS 1, COS 7 and CV1, quail QC1-3 cells, mouse L cells, mouse sarcoma cells, Bowes melanoma cells, human CAP or CAP-T cells and Chinese hamster ovary (CHO) cells. Also within the scope of the present present disclosure are primary mammalian cells or cell lines. Primary cells are cells which are directly obtained from an organism. Suitable primary cells are, for example, mouse embryonic fibroblasts (MEF), mouse primary hepatocytes, cardiomyocytes and neuronal cells as well as mouse muscle stem cells (satellite cells), human dermal and pulmonary fibroblasts, human epithelial cells (nasal, tracheal, renal, placental, intestinal, bronchial epithelial cells), human secretory cells (from salivary, sebaceous and sweat glands), human endocrine cells (thyroid cells), human adipose cells, human smooth muscle cells, human skeletal muscle cells, and stable, immortalized cell lines derived thereof (for example hTERT or oncogene immortalized cells).

The host cell in accordance with this embodiment may for example be employed in methods for the amplification of vectors of the present disclosure, for the production of the fusion protein of the present disclosure or for the direct production of lentivirus particles, as described in more detail herein below.

Suitable conditions for culturing a prokaryotic or eukaryotic host are well known to the person skilled in the art. For example, suitable conditions for culturing bacteria are growing them under aeration in Luria Bertani (LB) medium. To increase the yield and the solubility of the expression product, the medium can be buffered or supplemented with suitable additives known to enhance or facilitate both. E. coli can be cultured from 4 to about 37° C., the exact temperature or sequence of temperatures depends on the molecule to be over-expressed. In general, the skilled person is also aware that these conditions may have to be adapted to the needs of the host and the requirements of the protein expressed. In case an inducible promoter controls the nucleic acid of the present disclosure in the vector present in the host cell, expression of the polypeptide can be induced by addition of an appropriate inducing agent. Suitable expression protocols and strategies are known to the skilled person.

Depending on the cell type and its specific requirements, mammalian cell culture can, e.g., be carried out in RPMI, Williams' E or DMEM medium containing 10% (v/v) FCS, 2 mM L-glutamine and 100 U/ml penicillin/streptomycine. The cells can be kept, e.g., at 37° C. or at 41° C. for DT40 chicken cells, in a 5% CO2, water saturated atmosphere. Suitable media for insect cell culture is, e.g., TNM+10% FCS or SF900 medium. Insect cells are usually grown at 27° C. as adhesion or suspension culture. Suitable expression protocols for eukaryotic or vertebrate cells are well known to the skilled person and can be retrieved, e.g., from Sambrook and Russel, loc. cit.

A “lentiviral vector particle”, also referred to herein as a “lentiviral vector”, is a vector based on a lentivirus virion, i.e., a subclass of retroviruses that can integrate into the genome of non-dividing target cells. A unique feature of lentiviruses is that they have a self-inactivated (SIN) region of replication in contrast to other retroviral vectors. Lentiviruses are well known in the art and have been described in detail, e.g., in Retroviruses, Coffin J M, Hughes S H, Varmus H E, Cold Spring Harbor (N.Y.): Cold Spring Harbour Laboratory Press; 1997; ISBN-10:0-87969-571-4; O'Connell R M, Balazs A B, Rao D S, Kivork C, Yang L, Baltimore D. Lentiviral vector delivery of human interleukin-7 (hIL-7) to human immune system (HIS) mice expands T lymphocyte populations. PLOS One. 2010 Aug. 6; 5(8): e 12009; Mátrai J, Chuah M K, VandenDriessche T. Recent advances in lentiviral vector development and applications. Mol Ther. 2010 March; 18(3): 477-90.

A lentiviral vector particle can be based, e.g., on a lentivirus of the group of bovine, equine, feline, ovine/caprine or primate lentiviruses. In particular, the lentiviral vector is based on a primate lentivirus such as HIV1, HIV2 or SIV virus. More specifically, the lentiviral vector is based on an HIV1 lentivirus. As the skilled person is aware, most (commercially available) lentiviral vectors represent a mixture of viral constituents from different viruses and are, hence, to some extent “hybrid” vectors. For example, a lentiviral vector may comprise constituents from HIV1, VSVg, CMV, WPRE viruses. Such hybrid vectors are explicitly envisaged in accordance with the present disclosure.

The term “pseudotyped”, as used herein in the context of viral vectors, refers to the modulation of the cell type specificity of a viral vector by integration of foreign viral envelope proteins. This approach is well known in the art and has been described for example in Bischof et al. (Flexibility in cell targeting by pseudotyping lentiviral vectors. Methods Mol Biol. 2010; 614:53-68). Using this approach, host tropism can be altered and/or the stability of the virus can be decreased or increased. For example, the use of VSV-G for pseudotyping a lentiviral virus has been described, e.g., in Burns et al. (Vesicular stomatitis virus G glycoprotein pseudotyped retroviral vectors: concentration to very high titer and efficient gene transfer into mammalian and nonmammalian cells. Proc Natl Acad Sci USA. 1993; 90(17): 8033-8037).

The present disclosure further relates to a method of producing the pseudotyped lentiviral vector particle, the method comprising transfecting into a host cell (i) one or more packaging plasmids encoding the virion proteins and accessory proteins needed for efficient production and packaging of the LTR-containing nucleic acid; (ii) a vector comprising the nucleic acid molecule of the present disclosure; and (iii) a vector comprising a nucleic acid molecule encoding a VSV-G not linked to a (poly)peptide comprising or consisting of a cell membrane-binding domain.

Such methods of producing pseudotyped lentiviral vectors are well known in the art and have been described, e.g., in Naldini, L. (1998) [61]. Host cells, in particular HEK 293, HEK293T, CAP or CAP-T cells are employed and a number of vectors, including the packaging vector(s) encoding the viral proteins, such as e.g. the capsid and the reverse transcriptase, as well as vectors carrying the nucleic acid molecules to be additionally introduced into the pseudotyped lentiviral vector particle are transfected or electroporated into these cells, or nucleofection is used to transfer said vectors. In addition, further vectors containing the genetic material to be delivered by the pseudotyped lentiviral vector particle may be transfected.

These vectors may be introduced into the host cells by direct introduction or by introduction via electroporation (using for example Multiporator (Eppendorf), Genepulser (BioRad), MaxCyte Transfection Systems (Maxcyte)), PEI (Polysciences Inc. Warrington, Eppelheim), Ca²⁺-mediated transfection or via liposomes (for example: “Lipofectamine” (Invitrogen)), non-liposomal compounds (for example: “Fugene” (Roche) or nucleofection (Lonza)) into cells.

The present disclosure further relates to a method for transducing cells, the method comprising the step of contacting cells to be transduced with the pseudotyped lentiviral vector particle under conditions suitable for transduction, thereby transducing said cells.

The term “transducing”, as used herein, is well known in the art and refers to the process of introducing genetic material into a cell and, optionally, its subsequent integration into the genome of said cell via viral vector particles. Said genetic material comprises or consists of viral RNA combined with one or more target RNA sequences (hereinafter referred to as target sequences) comprised in said viral vector particles intended for integration into the genome of a target cell.

The term “contacting” as used herein in the context of this method of the present disclosure refers to bringing the cells to be transduced (also referred to herein as “target cells”) into contact with a retroviral vector so that the transduction event can occur. Conditions for contacting that allow the transduction event to occur are well known in the art and may depend to a certain extent on the cell to be transduced. For example, some target cells are more difficult to transfect than other cells and may need to be transitioned into a specific culture medium before transduction with a viral vector can be achieved. Corresponding methods and conditions are described for example in Jacome et al. (Lentiviral-mediated Genetic Correction of Hematopoietic and Mesenchymal Progenitor Cells From Fanconi Anemia Patients. Mol Ther. 2009 June; 17(6): 1083-1092), Chu et al. (Efficient and Stable Gene Expression into Human Osteoclasts Using an HIV-1—Based Lentiviral Vector. DNA Cell Biol. 2008 June; 27(6): 315-320), or Poczobutt et al. (Benign mammary epithelial cells enhance the transformed phenotype of human breast cancer cells. BMC Cancer. 2010; 10:373).

The cells to be transduced can be any cells of interest that are to be targeted for transduction with a viral vector, particularly non-transducible cells, such as with low LDLR. The term “cell/cells” as used herein can refer to single and/or isolated cells or to cells that are part of a multicellular entity such as a tissue, an organism or a cell culture. In other words the method can be performed in vivo, ex vivo or in vitro. In particular, the cells to be transduced are eukaryotic cells including any cell of a multi-cellular eukaryotic organism, in particular cells from animals like vertebrates. More specifically, the cells to be transduced are mammalian cell. Depending on the particular goal to be achieved through modifying the genome of a mammalian cell by transducing it according to the method of the present disclosure, cells of different mammalian subclasses such as prototheria or theria may be used. For example, within the subclass of theria, in particular cells of animals of the infraclass eutheria, more specifically of the order primates, artiodactyla, perissodactyla, rodentia and lagomorpha are used in the method of the present disclosure. Furthermore, within a species one may choose a cell to be used in the method of the present disclosure based on the tissue type and/or capacity to differentiate equally depending on the goal to be achieved by modifying the genome via transducing a target cell according to the method of the present disclosure. Three basic categories of cells, which in principle can be transduced with the method of the present disclosure, make up the mammalian body: germ cells, somatic cells and stem cells. A germ cell is a cell that gives rise to gametes and thus is continuous through the generations. Stem cells can divide and differentiate into diverse specialized cell types as well as self-renew to produce more stem cells. In mammals there are two main types of stem cells: embryonic stem cells and adult stem cells. Somatic cells include all cells that are not gametes, gametocytes or undifferentiated stem cells. The cells of a mammal can also be grouped by their ability to differentiate. A totipotent (also known as omnipotent) cell is a cell that is able to differentiate into all cell types of an adult organism including placental tissue such as a zygote (fertilized oocyte) and subsequent blastomeres, whereas pluripotent cells, such as embryonic stem cells, cannot contribute to extraembryonic tissue such as the placenta, but have the potential to differentiate into any of the three germ layers endoderm, mesoderm and ectoderm. Multipotent progenitor cells have the potential to give rise to cells from multiple, but limited number of cell lineages. Further, there are oligopotent cells that can develop into only a few cell types and unipotent cells (also sometimes termed a precursor cell) that can develop into only one cell type. There are four basic types of tissues: muscle tissue, nervous tissue, connective tissue and epithelial tissue that cells to be used in the method of the present disclosure can be derived from, such as for example lymphoid lineage cells or neuronal stem cells.

The term “lymphoid lineage cells” refers to cells that are involved in the generation of lymphocytes and lymphocytes per se. The term “lymphocyte” refers to small lymphocytes (B and T lymphocytes, plasma cells) and natural killer cells as well-known in the art. Lymphoid lineage cells further include, e.g., lymphoid dendritic cells, as well as lymphocyte progenitor cells such as pro-lymphocytes, lymphoblasts, common lymphoid progenitor cells.

The term “epithelial cell” is well known in the art. Epithelial cells line cavities and surfaces of structures throughout the body and also form many glands. Epithelial tissues can be classified into simple epithelium (one cell thick) and stratified epithelium (several layers of cells). Epithelial cells are furthermore classified by their morphology into squamous, cuboidal, columnar and pseudostratified epithelial cells. For example, the human stomach and intestine is lined with epithelial cells. Further, epithelial cell lines include also breast carcinoma cells (such as, e.g., MCF7, MDA-MB-361 and T47D cells) or cells of the cell line HEK293T.

The term “adjuvant”, as used herein, relates to a compound that enhances the efficiency of the lentiviral transduction. Non-limiting examples of adjuvants include, e.g., a poloxamer having a molecular weight of 12.8 kDa to about 15 kDa and polybrene.

The term “poloxamer” is well known in the art and refers to a non-ionic triblock copolymer composed of a central hydrophobic chain of polyoxypropylene flanked by two hydrophilic chains of polyoxyethylene.

The lentiviral vector particles and the adjuvant can be added simultaneously, e.g., as a mixture, to the target cells or in sequential mode, as long as both compounds are simultaneously in contact with the target cell to allow transduction.

### III. Methods of Use

The present disclosure provides methods of providing gene therapy and lentivirus-based in vitro gene delivery, human naïve T cell engineering, and genetic screens using murine T cells for T cell immunology and cancer immunology research.

Gene therapy can be performed by gene transfer, gene editing (e.g., CRISPR), exon skipping, RNA-interference, trans-splicing or any other genetic modification of any coding or regulatory sequences in the cell, including those included in the nucleus, mitochondria or as commensal DNA (viral sequences contained in cells).

The two main types of gene therapy are the following: a therapy aiming the replacement of a deficient/abnormal gene: this is replacement gene therapy; or a therapy aiming gene editing: in such a case, the purpose is to provide to a cell the necessary tools so that the gene of interest is expressed: this is gene editing therapy.

In replacement gene therapy, the gene of interest may be a correct version of a gene which is deficient or mutated in a patient, as is the case for example in a genetic disease. In such a case, the gene of interest will restore the expression of the deficient or mutated gene. Of particular interest are deficient or mutated genes in patients exhibiting a disease which, once corrected in immune cells, in particular in B cells, T cells, monocytes or dendritic cells, more specifically in B cells, improve the patient's disease or symptoms.

In gene therapy, it might be possible to use the present pseudotyped lentiviral particles and chemically labeled cells in therapy for immune cell engineering, in particular T or B cell engineering, by transducing said immune cells. It might be possible to generate regulatory B cells by expressing immunosuppressive proteins in B cells, for instance IL-10, or to induce the production of immunoregulatory proteins such as antibodies or fusion proteins.

It could also be possible to insert sequences favoring gene splicing, expression or regulation or gene editing. Tools such as CRISPR/Cas9 may be used for this purpose. This could be used to modify gene expression in T or B cells, in the case of autoimmunity or cancer, or to perturb the cycle of viruses in B cells. In such cases, in particular, the heterologous gene of interest is chosen from gRNA, nucleases, DNA templates and RNAi components, such as shRNA.

An immune cell is a cell involved in the immune system. It comprises notably B cells, T cells, NK cells, macrophages and dendritic cells. A B cell (or B lymphocyte) is an immune cell responsible for the production of antibodies and involved in the humoral immune response. A dendritic cell is an immune cell, which is accessory, i.e., it is an antigen-presenting cell. Its main function is to process the antigen material and present it on its cell surface to the T cells. A T cell (or T lymphocyte) is an immune cell involved in cell-mediated immune response. It may be chosen from killer T cells, helper T cells and gamma delta T cells.

The term “subject” or “patient” as used herein refers to any individual to which the subject methods are performed. Generally, the patient is human, although as will be appreciated by those skilled in the art, the patient may be an animal. Thus, other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of patient.

“Treatment” and “treating” refer to administration or application of a therapeutic agent to a subject or performance of a procedure or modality on a subject for the purpose of obtaining a therapeutic benefit of a disease or health-related condition. For example, a treatment may include administration chemotherapy, immunotherapy, radiotherapy, performance of surgery, or any combination thereof.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the invasiveness of a tumor, reduction in the growth rate of the cancer, or prevention of metastasis. Treatment of cancer may also refer to prolonging survival of a subject with cancer.

### IV. Examples

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Click-Chemistry Modified LDLR or I1 Antibody

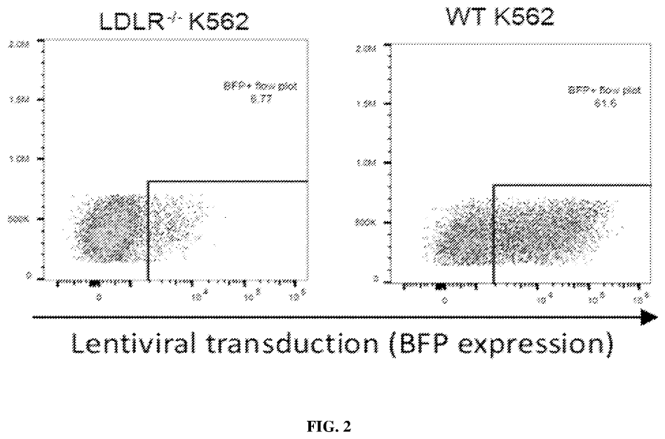

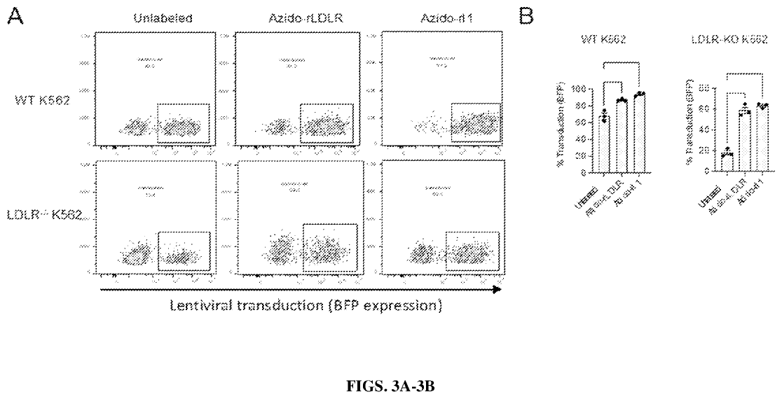

First, a K562 cell line was developed with hLDLR knocked out via CRISPR-Cas9 to model various cell types with low hLDLR expression. The CRISPR Cas9 cassette targeting the hLDLR gene locus was transduced into K 562 cells via lentiviral vector. After 6 days of selection by puromycin, the transduction efficiency of the hLDLR KO cells using BFP-expressing standard VSV-G pseudotyped lentivirus. In these hLDLR KO cells, transduction by VSV-G pseudotyped lentivirus is only 7% (FIG. 2), from a typical transduction efficiency of about 60% (FIG. 3A). This shows that transduction efficiency correlates with the presence of the hLDLR in K562 cells, confirming that a lack of hLDLR results in a lower transduction.

To demonstrate that the lentivirus entry receptor can be chemically restored to target cells, a click-chemistry based two-step labeling procedure was performed: 1) DBCO was randomly conjugated to the recombinant LDLR or the I1 antibody (specifically binds to VSV-G) using Sulfo-NHS DBCO and 2) azide was conjugated to the cell surface proteins using 1 μM 6-azidohexanoic acid-NHS ester (FIG. 1). The proteins were then clicked to the cells by mixing the DBCO-labeled protein with Azide-labeled cells by mixing them at 0.1 mg/ml and incubating at 37° C. for 30 min. Cells were washed 2× with 1× PBS and transduced with lentivirus expressing BFP (blue fluorescent protein) by centrifuging cells with virus at 2,000 g for 90 min at 35° C. Over 80% transduction was seen for both conjugating the I1 antibody to the cell surface, and by conjugating LDLR to the cell surface. The difference between unlabeled and protein-conjugated cells was greater for KO LDLR K562 cells, as wt K562 cells have endogenous LDLR that already binds VSV-G and facilitate high levels of transduction (FIG. 3)

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### V. References

The following references, to the extent that they provide exemplary procedural or other details supplementary to those set forth herein, are specifically incorporated herein by reference.
Bischof et al., Flexibility in cell targeting by pseudotyping lentiviral vectors. Methods Mol Biol. 2010; 614:53-68.
Braasch and Corey, Chemistry & Biology 8, 1-7 (2001)
Chu et al., Efficient and Stable Gene Expression into Human Osteoclasts Using an HIV-1—Based Lentiviral Vector. DNA Cell Biol. 2008 June; 27(6): 315-320.
Coffin J M, Hughes S H, Varmus H E, Cold Spring Harbor (N.Y.): Cold Spring Harbour Laboratory Press; 1997; ISBN-10:0-87969-571-4.
Egholm et al., Nature 365:666 (1993).
Jacome et al., Lentiviral-mediated Genetic Correction of Hematopoietic and Mesenchymal Progenitor Cells from Fanconi Anemia Patients. Mol Ther. 2009 June; 17(6): 1083-1092.
Mátrai J, Chuah M K, VandenDriessche T. Recent advances in lentiviral vector development and applications. Mol Ther. 2010 March; 18(3): 477-90.
Nielsen et al., Science 254:1497 (1991).
O'Connell R M, Balazs A B, Rao D S, Kivork C, Yang L, Baltimore D. Lentiviral vector delivery of human interleukin-7 (hIL-7) to human immune system (HIS) mice expands T lymphocyte populations. PLoS One. 2010 August 6; 5(8): e 12009.
Poczobutt et al., Benign mammary epithelial cells enhance the transformed phenotype of human breast cancer cells. BMC Cancer. 2010; 10:373.

1. A method of chemically labeling a cell comprising attaching to the surface of said cell a recombinant low density lipoprotein receptor (LDLR) protein or anti-vesicular stomatitis virus envelope glycoprotein (VSV-G) antibody or fragment thereof by performing click chemistry.

2. The method of claim 1, wherein the cell has low, undetectable or essentially no expression of LDLR.

3. The method of claim 1, wherein the anti-VSV-G antibody is I1, M55, 8G5F11 or IE9F9 antibody.

4. The method of claim 1, wherein performing click chemistry comprises:
(a) conjugating Sulfo-NHS DBCO to recombinant LDLR or anti-VSV-G antibody to obtain DBCO-labeled protein;
(b) conjugating azide to cell surface proteins of said cell using 6-azidohexanoic acid-NHS ester to obtain an azide-labeled cell; and
(c) mixing the DBCO-protein with the azide-labeled cell to obtain a LDLR or anti-VSV-G antibody labeled cell.

5. The method of claim 4, wherein the mixing is performed at 0.1 mg/mL.

6. The method of claim 2, wherein the cell with low or essentially no expression of hLDLR is a human naïve T cell, a resting human T cell, a human B cell, a human epithelial cell, a murine immune cell, a canine immune cell, a monkey immune cell, or a cancer cell line.

7. The method of claim 6, wherein the murine immune cell, monkey immune cell, or canine immune cell is a T cell, NK cell, or hematopoietic cell.

8. The method of claim 6, wherein the murine immune cell, monkey immune cell, or canine immune cell is a T cell.

9. The method of claim 6, wherein the cancer cell line is SUP-B15 or Raji.

10. The method of claim 1, further comprising transducing the cell with lentivirus.

11. The method of claim 10, wherein the lentivirus comprises VSV-G as the envelope protein.

12. The method of claim 10, wherein transduction efficiency is at least 80%.

13. A chemically labeled cell having a recombinant LDLR or anti-VSV-G antibody or fragment thereof attached to the surface.

14. The cell of claim 13, wherein the cell is produced by the method of claim 1.

15. The cell of claim 13, wherein the cell has been transduced by a VSV-G lentivirus.

16. (canceled)
